(19) United States
(12) Patent Application Publication
(10) Pub. No.: US 2021/0298848 A1
(43) Pub. Date: Sep. 30, 2021

(54) ROBOTICALLY-ASSISTED SURGICAL DEVICE, SURGICAL ROBOT, ROBOTICALLY-ASSISTED SURGICAL METHOD, AND SYSTEM

(71) Applicants: Medicaroid Corporation, Kobe-shi (JP); Ziosoft, Inc., Tokyo (JP)

(72) Inventors: Jota IDA, Kobe-shi (JP); Mitsuichi HIRATSUKA, Kobe-shi (JP); Shusuke CHINO, Tokyo (JP); Tsuyoshi NAGATA, Tokyo (JP); Yutaka KARASAWA, Tokyo (JP); Shinichiro SEO, Tokyo (JP)

(21) Appl. No.: 17/211,966

(22) Filed: Mar. 25, 2021

(30) Foreign Application Priority Data
Mar. 26, 2020 (JP) 2020-055966

(51) Int. Cl. A61B 34/30; G16H 20/40; G16H 30/20

(52) U.S. Cl. CPC A61B 34/30; A61B 2090/065; G16H 30/20; G16H 20/40

(57) ABSTRACT

A robotically-assisted surgical device assists robotic surgery by a surgical robot. The robotically-assisted surgical device includes a processor. The processor is configured to: acquire 3D data of a subject; acquire a contact position where a surgical instrument provided in the surgical robot is in contact with a soft tissue of the subject; acquire firmness of the contact position of the soft tissue of the subject; and perform registration of a position of the 3D data with a position of the subject recognized by the surgical robot according to deformation of the soft tissue in the 3D data, based on the contact position and the firmness.

### CROSS-REFERENCE TO RELATED APPLICATIONS

This application is based on and claims priority under 35 USC 119 from Japanese Patent Application No. 2020-055966 filed on Mar. 26, 2020, the contents of which are incorporated herein by reference.

### TECHNICAL FIELD

The present disclosure relates to a robotically-assisted surgical device, a surgical robot, a robotically-assisted surgical method, and a system.

### BACKGROUND ART

In the related art, robotic surgery has been performed for patients using surgical robots. It is known that, in robotic surgery, a contact sensor detects contact with the bone of the patient and the position of the patient is registered based on the detection result (refer to Japanese Unexamined Patent Application Publication No. 2018-126498).

Transanal minimally invasive surgery (TAMIS) is known as one of the surgical procedures. In TAMIS, it is known to install a platform (Transanal Access Platform) in an anus of a patient in order to insert a surgical instrument into the patient (refer to GelPOINT Path, Transanal Access Platform, Applied Medical, searched on Dec. 26, 2019, Internet <URL: https://www.appliedmedical.com/Products/Gelpoint/Path>).

It is difficult to apply registering methods, which are based on the detection result of contact with hard tissues such as bones by contact sensors in the related art, to soft tissues that are easily deformed.

For example, in TAMIS, the tissues in the subject are easily moved and rotated according to the body position change of the subject, and the deformation of the tissue is likely to occur. There is also a case where the tissue is deformed as the surgical instrument comes into contact with tissues in the subject during surgery. Before surgery, in order to observe the condition of the subject, the subject is imaged by a CT scanner or the like, and the volume data of the subject is prepared.

Here, even when the tissues in the subject in the actual space are deformed during surgery, the volume data in the virtual space will not be deformed. Therefore, a gap arises between the position of the subject in the actual space and the position indicated by the volume data in the virtual space. This gap can deteriorate safety in endoscopic surgery.

Regarding hard tissues such as bones, when the position of the subject and the position indicated by the volume data of the virtual space are once registered, both positions are not likely to shift thereafter.

On the other hand, soft tissues, such as a rectum, are easily affected by the movement of the subject or contact with surgical instruments, and are easily deformed, and thus, the need for registration is particularly high.

In robotic surgery, the sense of touch is limited for the operator, and particularly when there are different tissues which are easily deformed and behind the soft tissue, it is difficult to grasp the tissue behind the soft tissue by the sense of touch.

In view of the above-described circumstances, the present disclosure provides a robotically-assisted surgical device, a surgical robot, a robotically-assisted surgical method, and a program that can easily register an actual position of a subject with a position of 3D data of the subject, taking into account soft tissues that are easily deformed.

### SUMMARY

A robotically-assisted surgical device related to one aspect of the present disclosure assists robotic surgery by a surgical robot. The robotically-assisted surgical device includes a processor. The processor is configured to: acquire 3D data of a subject; acquire a contact position where a surgical instrument provided in the surgical robot is in contact with a soft tissue of the subject; acquire firmness of the contact position of the soft tissue of the subject; and perform registration of a position of the 3D data with a position of the subject recognized by the surgical robot according to deformation of the soft tissue in the 3D data, based on the contact position and the firmness.

According to the present disclosure, the actual position of the subject and the position of the model of the subject can be easily registered, taking into account the soft tissues that are easily deformed.

### BRIEF DESCRIPTION OF DRAWINGS

Exemplary embodiment(s) of the present invention will be described in detail based on the following figures, wherein:

FIG. 1 is a block diagram illustrating a configuration example of a robotically-assisted surgical system according to a first embodiment;

FIG. 2 is a block diagram illustrating a hardware configuration example of a robotically-assisted surgical device;

FIG. 3 is a block diagram illustrating a functional configuration example of the robotically-assisted surgical device;

FIG. 4 is a view illustrating an example of a state of a platform, a surgical instrument, and the inside of a subject;

FIG. 5A is a view illustrating an example of a state of pelvis in a state where a body position of the subject is a lithotomy position and a leg part is raised low;

FIG. 5B is a view illustrating an example of a slate of the pelvis in a state where the body position of the subject is the lithotomy position and the leg part is raised low;

FIG. 6A is a schematic view illustrating an example in which there is a bone behind an intestinal wall with which a contact sensor is in contact;

FIG. 6B is a schematic view illustrating an example in which there is no bone behind the intestinal wall with which the contact sensor is in contact;

FIG. 6C is a schematic view illustrating an example in which there is the bone a little apart behind the intestinal wall with which the contact sensor is in contact;

FIG. 7A is a schematic view illustrating an example of a state before the contact sensor comes into contact with tendon with which the contact sensor is in contact;

FIG. 7B is a schematic view illustrating an example of a state where the contact sensor is in contact with the tendon with which the contact sensor is in contact;

FIG. 8 is a schematic view illustrating an example in which there is an intestinal wall behind the intestinal wall with which the contact sensor is in contact;

FIG. 9 is a flowchart illustrating an operation example of the robotically-assisted surgical device; and

FIG. 10 is a flowchart illustrating an operation example of the robotically-assisted surgical device (continued from FIG. 9).

### DESCRIPTION OF EMBODIMENTS

Hereinafter, embodiments of the present disclosure will be described with reference to the drawings.

### First Embodiment

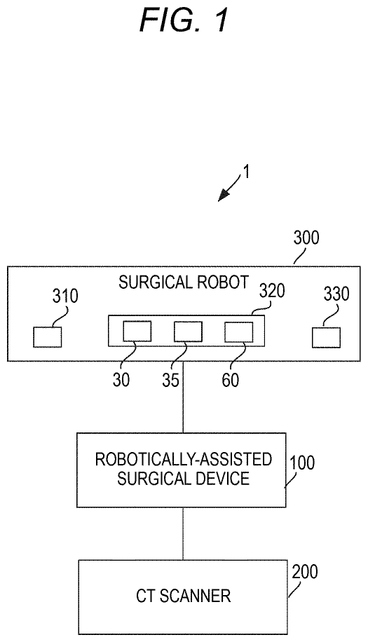

FIG. 1 is a block diagram illustrating a configuration example of a robotically-assisted surgical system 1 according to a first embodiment. The robotically-assisted surgical system 1 includes a robotically-assisted surgical device 100, a CT scanner 200, and a surgical robot 300. The robotically-assisted surgical device 100, the CT scanner 200, and the surgical robot 300 may be connected to each other via a network. The robotically-assisted surgical device 100 may be connected to each device of the CT scanner 200 and the surgical robot 300 on a one-to-one basis. FIG. 1 exemplifies that the robotically-assisted surgical device 100 is connected to each of the CT scanner 200 and the surgical robot 300.

The robotically-assisted surgical device 100 acquires various pieces of data from the CT scanner 200 and the surgical robot 300. The robotically-assisted surgical device 100 performs image processing based on the acquired data to assist the robotic surgery by the surgical robot 300. The robotically-assisted surgical device 100 may be configured of a PC and software installed in the PC. The robotically-assisted surgical device 100 performs surgery navigation. The surgery navigation includes, for example, preoperative simulation for performing planning before surgery (preoperative planning) and intraoperative navigation for performing the assistance during surgery.

The CT scanner 200 irradiates the subject with X-rays, and captures images (CT images) by using the difference in X-ray absorption by tissues in the body. The subject may include a living body, a human body, an animal, and the like. The CT scanner 200 generates the volume data including information on any location on the inside of the subject. The CT scanner 200 transmits the volume data as the CT image to the robotically-assisted surgical device 100 via a wired circuit or a wireless circuit. Imaging conditions for CT images or contrast conditions for administration of a contrast medium may be taken into consideration when capturing CT images.

The surgical robot 300 includes a robot operation terminal 310, a robot main body 320, and an image display terminal 330.

The robot operation terminal 310 includes a hand controller and a foot switch operated by an operator. The robot operation terminal 310 operates a plurality of robot arms AR provided in the robot main body 320 in response to the operation of the hand controller or the foot switch by the operator. The robot operation terminal 310 includes a viewer. The viewer may be a stereo viewer, and may display a three-dimensional image by fusing the images captured by an endoscope ES (endoscope camera). The plurality of robot operation terminals 310 may exist, and the robotic surgery may be performed by a plurality of operators operating the plurality of robot operation terminals 310.

The robot main body 320 includes the plurality of robot arms AR for performing the robotic surgery, an end effector EF (forceps, instruments) attached to the robot arm AR, and the endoscope ES attached to the robot arm AR. Since the end effector EF and the endoscope ES are used for endoscopic surgery, the end effector EF and the endoscope ES are also referred to as surgical instruments 30 in the embodiment. The surgical instrument 30 includes at least one of one or more end effectors EF and endoscopes ES.

The robot main body 320 is provided with, for example, four robot arms AR, and includes a camera arm to which the endoscope ES is attached, a first end effector arm to which the end effector EF operated by the hand controller for the right hand of the robot operation terminal 310 is attached, a second end effector arm to which the end effector EF operated by the hand controller for the left hand of the robot operation terminal 310 is attached, and a third end effector arm to which the end effector EF for the replacement is attached. Each robot arm AR has a plurality of joints, and may be provided with a motor and an encoder corresponding to each joint. The encoder may include a rotary encoder as an example of an angle detector. Each robot arm AR has at least 6 degrees of freedom, preferably 7 or 8 degrees of freedom, and may operate in the three-dimensional space and be movable in each direction within the three-dimensional space. The end effector EF is an instrument that actually comes into contact with the treatment target in a subject PS in the robotic surgery, and enables various treatments (for example, grasping, excision, peeling, and suturing).

The end effector EF may include, for example, grasping forceps, peeling forceps, an electric knife, and the like. As the end effector EF, a plurality of separate end effector EFs different for each role may be prepared. For example, in the robotic surgery, the tissue may be suppressed or pulled by two end effector EFs, and the tissue may be cut by one end effector EF. The robot arm AR and the surgical instrument 30 may operate based on an instruction from the robot operation terminal 310. At least two end effectors EF are used in the robotic surgery.

The robot main body 320 includes a processing unit 35 and a contact sensor 60. The processing unit 35 is configured with a processor, for example. The processor functions as the processing unit 35 that performs various types processing and control by executing a program stored in a memory provided in the robot main body 320.

The contact sensor 60 may, for example, be installed on the surgical instrument 30 (for example, end effector EF) and may be installed at the distal end of the surgical instrument 30. The contact sensor 60 detects the presence or absence of contact with the soft tissue in the subject PS. The processing unit 35 transmits contact detection information including the information on the presence or absence of contact detected by the contact sensor 60, to the robotically-assisted surgical device 100 via a communication unit (wired communication unit or wireless communication unit) provided in the robot main body 320. The contact sensor 60 may detect the contact position where the contact sensor 60 (for example, the distal end of the surgical instrument 30) comes into contact with the soft tissue in the subject PS. The contact detection information may include information on the contact position.

The contact sensor 60 may also operate as a pressure sensor. In other words, the contact sensor 60 may detect the magnitude of the reaction force received from the soft tissue which is in contact the contact sensor 60. The contact detection information may include the information on the reaction force detected by the contact sensor 60. The contact sensor 60 and the pressure sensor may be installed separately as different sensors instead of being integrated.

Soft tissues in the subject PS are tissues other than hard tissues such as bones, and may include intestines (intestinal wall), muscles, blood vessels, and the like. Unlike hard tissues such as bones, soft tissues move easily, for example, move easily during surgery due to body position changes or contact with the surgical instrument 30. The movement of the soft tissue also affects the tissue neighbor of the soft tissue. Therefore, it is advantageous to perform registration processing according to the deformation of the soft tissue as compared with the hard tissue.

The image display terminal 330 has a monitor and a controller for processing the image captured by the endoscope ES and displaying the image on a viewer or a monitor. The monitor is confirmed by, for example, a robotic surgery assistant or a nurse.

The surgical robot 300 performs the robotic surgery in which an operation of the hand controller or the foot switch of the robot operation terminal 310 by the operator is received, the operations of the robot arm AR, the end effector EF, and the endoscope ES of the robot main body 320 are controlled, and various treatments for the subject PS are performed. In the robotic surgery, the endoscopic surgery may be performed in the subject PS.

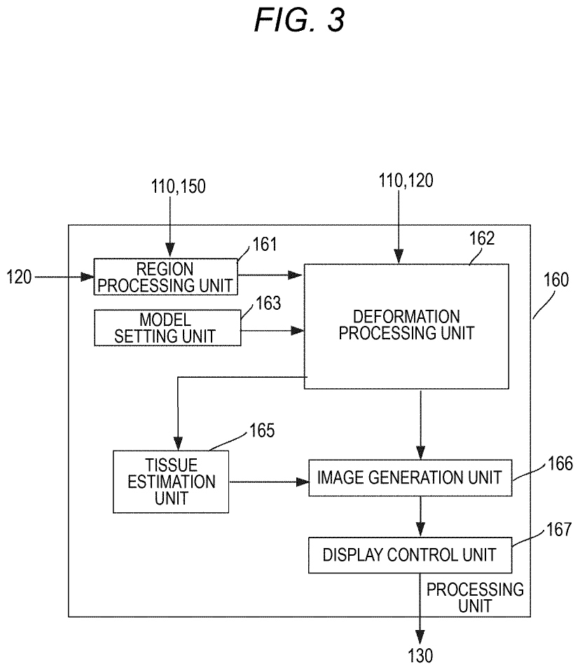

In the embodiment, it is mainly assumed that Transanal Minimally Invasive Surgery (TAMIS) is performed using the surgical robot 300. TAMIS is one type of endoscopic surgery using a natural opening portion. In TAMIS, a platform 40 (Transanal Access Platform) (refer to FIG. 3) is installed on the anus of the subject PS in order to insert the surgical instrument 30 into the subject PS. In TAMIS, since the platform 40 is installed on the anus, which is a hole of the subject PS, it is not necessary to perforate a port on the body surface of the subject PS unlike installation of a trocar. In TAMIS, gas may be injected through the platform 40 to inflate the tissues or organs existing in the neighborhood of the anus of the subject PS. In TAMIS, the body position of the subject PS is, for example, a lithotomy position, but other body positions (for example, jackknife position) may be employed. The tissues or organs existing in the neighborhood of the anus of the subject PS may include, for example, rectum, colon, prostate, and the like. The platform 40 has a valve and maintains the inside of the subject PS airtight. Air (for example, carbon dioxide) may be continuously introduced into the subject PS for maintaining the airtight state.

The end effector EF is inserted through the platform 40. The valve of the platform 40 is opened when the end effector EF is inserted, and the valve of the platform 40 is closed when the end effector EF is detached. The end effector EF is inserted via the platform 40, and various treatments are performed depending on the surgical procedure. The robotic surgery may be applied to the endoscopic surgery (for example, palatal jaw surgery, mediastinal surgery, and laparoscopic surgery) of other parts in addition to a case where the organs neighbor of the anus are surgery targets.

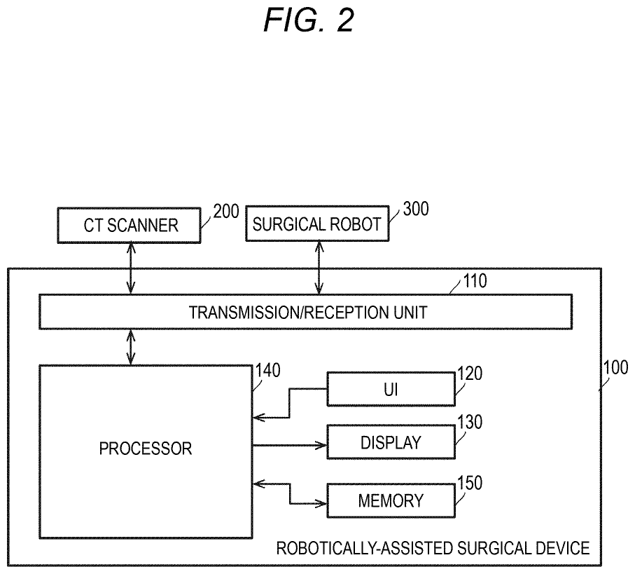

FIG. 2 is a block diagram illustrating a configuration example of the robotically-assisted surgical device 100. The robotically-assisted surgical device 100 includes a transmission/reception unit 110, a UI 120, a display 130, a processor 140, and a memory 150.

The transmission/reception unit 110 includes a communication port, an external device connection port, a connection port to an embedded device, and the like. The transmission/reception unit 110 acquires various pieces of data from the CT scanner 200 and the surgical robot 300. The various pieces of acquired data may be immediately sent to the processor 140 (a processing unit 160) for various types of processing, or may be sent to the processor 140 for various types of processing when necessary after being stored in the memory 150. The various pieces of data may be acquired via a recording medium or a storage medium.

The transmission/reception unit 110 transmits and receives various pieces of data and from to the CT scanner 200 and the surgical robot 300. The various pieces of data to be transmitted may be directly transmitted from the processor 140 (the processing unit 160), or may be transmitted to each device when necessary after being stored in the memory 150. The various pieces of data may be sent via a recording medium or a storage medium.

The transmission/reception unit 110 may acquire volume data from the CT scanner 200. The volume data may be acquired in the form of intermediate data, compressed data or sinogram. The volume data may be acquired from information from a sensor device attached to the robotically-assisted surgical device 100.

The transmission/reception unit 110 acquires information from the surgical robot 300. The information from the surgical robot 300 may include information on the kinematics of the surgical robot 300. The information on the kinematics may include, for example, shape information regarding the shape and motion information regarding motion of an instrument (for example, the robot arm AR, the end effector EF, the endoscope ES) for performing the robotic surgery included in the surgical robot 300. The information on the kinematics may be received from an external server.

The shape information may include at least a part of information such as the length and weight of each part of the robot arm AR, the end effector EF, and the endoscope ES, the angle of the robot arm AR with respect to the reference direction (for example, a horizontal surface), and the attachment angle of the end effector EF with respect to the robot arm AR.

The motion information may include the movable range in the three-dimensional space of the robot arm AR, the end effector EF, and the endoscope ES. The motion information may include information such as the position, speed, acceleration, or orientation of the robot arm AR when the robot arm AR operates. The motion information may include information such as the position, speed, acceleration, or orientation of the end effector EF with respect to the robot arm AR when the end effector EF operates. The motion information may include information such as the position, speed, acceleration, or orientation of the endoscope ES with respect to the robot arm AR when the endoscope ES operates.

In the kinematics, together with the movable range of the robot arm itself, the movable range of the other robot arm is defined. Therefore, as the surgical robot 300 operates each robot arm AR of the surgical robot 300 based on the kinematics, it is possible to avoid interference of the plurality of robot arms AR with each other during surgery.

An angle sensor may be attached to the robot arm AR, the end effector EF, or the endoscope ES. The angle sensor may include a rotary encoder that detects an angle corresponding to the orientation of the robot arm AR, the end effector EF, or the endoscope ES in the three-dimensional space. The transmission/reception unit 110 may acquire the detection information detected by various sensors attached to the surgical robot 300. These various sensors may include the contact sensors 60.

The transmission/reception unit 110 may acquire operation information regarding the operation with respect to the robot operation terminal 310. The operation information may include information such as an operation target (for example, the robot arm AR, the end effector EF, the endoscope ES), an operation type (for example, movement, rotation), an operation position, and an operation speed.

The transmission/reception unit 110 may acquire surgical instrument information regarding the surgical instrument 30. The surgical instrument information may include the insertion distance of the surgical instrument 30 to the subject PS. The insertion distance corresponds, for example, to the distance between the platform 40 into which the surgical instrument 30 is inserted and the distal end position of the surgical instrument 30. For example, the surgical instrument 30 may be provided with a scale indicating the insertion distance of the surgical instrument 30. The transmission/reception unit 110 may electronically read the scale to obtain the insertion distance of the surgical instrument 30. In this case, for example, a linear encoder (reading device) may be attached to the platform 40, and the surgical instrument 30 may be provided with an encoding marker. The transmission/reception unit 110 may acquire the insertion distance of the surgical instrument 30 as the operator reads the scale and inputs the insertion distance via the UI 120.

The information from the surgical robot 300 may include information regarding the imaging by the endoscope ES (endoscopic information). The endoscopic information may include an image captured by the endoscope ES (actual endoscopic image) and additional information regarding the actual endoscopic image (imaging position, imaging orientation, imaging viewing angle, imaging range, imaging time, and the like).

The UI 120 may include, for example, a touch panel, a pointing device, a keyboard, or a microphone. The UI 120 receives any input operation from the operator of the robotically-assisted surgical device 100. Operators may include doctors, nurses, radiologists, students, and the like.

The UI 120 receives various operations. For example, an operation, such as designation of a region of interest (ROI) or setting of a brightness condition (for example, window width (WW) or window level (WL)), in the volume data or in an image (for example, a three-dimensional image or a two-dimensional image which will be described later) based on the volume data, is received. The ROI may include regions of various tissues (for example, blood vessels, organs, viscera, bones, and brain). The tissue may include diseased tissue, normal tissue, tumor tissue, and the like.

The display 130 may include an LCD, for example, and displays various pieces of information. The various pieces of information may include a three-dimensional image and a two-dimensional image obtained from the volume data. The three-dimensional images may include a volume rendering image, a surface rendering image, a virtual endoscopic image, a virtual ultrasound image, a CPR image, and the like. The volume rendering images may include a RaySum image, an MW image, a MinIP image, an average value image, a raycast image, and the like. The two-dimensional images may include an axial image, a sagittal image, a coronal image, an MPR image, and the like.

The memory 150 includes various primary storage devices such as ROM and RAM. The memory 150 may include a secondary storage device such as HDD or SSD. The memory 150 may include a tertiary storage device such as a USB memory, an SD card, or an optical disk. The memory 150 stores various pieces of information and programs. The various pieces of information may include volume data acquired by the transmission/reception unit 110, images generated by the processor 140, setting information set by the processor 140, and various programs. The memory 150 is an example of a non-transitory recording medium in which a program is recorded.

The processor 140 may include a CPU, a DSP, or a GPU. The processor 140 functions as the processing unit 160 that performs various types of processing and controls by executing the program stored in the memory 150.

FIG. 3 is a block diagram illustrating a functional configuration example of the processing unit 160. The processing unit 160 includes a region processing unit 161, a deformation processing unit 162, a model setting unit 163, a tissue estimation unit 165, an image generation unit 166, and a display control unit 167. Each unit included in the processing unit 160 may be realized as different functions by one piece of hardware, or may be realized as different functions by a plurality of pieces of hardware. Each unit included in the processing unit 160 may be realized by a dedicated hardware component.

The region processing unit 161 acquires the volume data of the subject PS via the transmission/reception unit 110, for example. The region processing unit 161 extracts any region included in the volume data. The region processing unit 161 may automatically designate the ROI and extract the ROI based on a pixel value of the volume data, for example. The region processing unit 161 may manually designate the ROI and extract the ROI via the UI 120, for example. The ROI may include regions such as organs, bones, blood vessels, affected parts (for example, diseased tissue or tumor tissue). Organs may include rectum, colon, prostate, and the like.

The ROI may be segmented (divided) and extracted including not only a single tissue but also tissues around the tissue. For example, in a case where the organ which is the ROI is the rectum, not only the rectum itself, but also blood vessels that are connected to the rectum or run in or in the neighborhood of the rectum, bones (for example, spine, pelvis) or muscles neighbor of the rectum, may also be included. The above-described rectum itself, the blood vessels in or in the neighborhood of the rectum, and the bones or muscles neighbor of the rectum may be segmented and obtained as separate tissues.

The model setting unit 163 sets a model of the tissue. The model may be set based on the ROI and the volume data. The model visualizes the tissue visualized by the volume data in a simpler manner than the volume data. Therefore, the data amount of the model is smaller than the data amount of the volume data corresponding to the model. The model is a target of deformation processing and deforming operation imitating various treatments in surgery, for example. The model may be, for example, a simple bone deformation model. In this case, the model deforms the bone by assuming a frame in a simple finite element and moving the vertices of the finite element. The deformation of the tissue can be visualized by following the deformation of the bone. The model may include an organ model imitating an organ (for example, rectum). The model may have a shape similar to a simple polygon (for example, a triangle), or may have other shapes. The model may be, for example, a contour line of the volume data indicating an organ. The model may be a three-dimensional model or a two-dimensional model. The bone may be visualized by the deformation of the volume data instead of the deformation of the model. This is because, since the bone has a low degree of freedom of deformation, visualization is possible by affine deformation of the volume data.

The model setting unit 163 may acquire the model by generating the model based on the volume data. A plurality of model templates may be predetermined and stored in the memory 150 or an external server. The model setting unit 163 may acquire a model by acquiring one model template among a plurality of model templates prepared in advance from the memory 150 or the external server in accordance with the volume data.

The model setting unit 163 may set the position of a target TG in the tissue (for example, an organ) of the subject PS included in the volume data. Otherwise, the model setting unit 163 may set the position of the target TG in the model imitating the tissue. The target TG is set in any tissue. The model setting unit 163 may designate the position of the target TG via the UI 120. The position of the target TG (for example, affected part) treated in the past for the subject PS may be stored in the memory 150. The model setting unit 163 may acquire and set the position of the target TG from the memory 150. The model setting unit 163 may set the position of the target TG depending on the surgical procedure. The surgical procedure indicates a method of surgery for the subject PS. The target position may be the position of the region of the target TG having a certain size. The target TG may be an organ that is subjected to sequential treatments by the surgical instrument 30 before reaching the affected part.

The surgical procedure may be designated via the UI 120. Each treatment in the robotic surgery may be determined by the surgical procedure. Depending on the treatment, the end effector EF required for the treatment may be determined. Accordingly, the end effector EF attached to the robot arm AR may be determined depending on the surgical procedure, and it may be determined which type of end effector EF is attached to which robot arm AR.

The deformation processing unit 162 performs processing related to the deformation in the subject PS which is a surgery target. For example, the tissue of an organ or the like in the subject PS can be subjected to various deforming operations by the operator by imitating various treatments performed by the operator in surgery. The deforming operation may include an operation of lifting an organ, an operation of flipping an organ, an operation of cutting an organ, and the like. In response to this, the deformation processing unit 162 deforms the model corresponding to the tissue of an organ or the like in the subject PS. For example, an organ can be pulled, pushed, or cut by the end effector EF, but may be simulated by deforming the model in this manner. When the model deforms, the targets in the model may also deform. The deformation of the model may include movement or rotation of the model.

The deformation by the deforming operation may be performed with respect to the model and may be a large deformation simulation using the finite element method. For example, movement of an organ due to the body position change may be simulated. In this case, the elastic force applied to the contact point of the organ or the disease, the rigidity of the organ or the disease, and other physical characteristics may be taken into consideration. In the deformation processing with respect to the model, the computation amount is reduced as compared with the deformation processing with respect to the volume data. This is because the number of elements in the deformation simulation is reduced. The deformation processing with respect to the model may not be performed, and the deformation processing may be directly performed with respect to the volume data.

The deformation processing unit 162 may perform the gas injection simulation in which gas is virtually injected into the subject PS through the anus, for example, as processing related to the deformation. The specific method of the gas injection simulation may be a known method, and for example, a pneumoperitoneum simulation method described in Reference Non-Patent Literature 1 (Takayuki Kitasaka, Kensaku Mori, Yuichiro Hayashi, Yasuhito Suenaga, Makoto Hashizume, and Jun-ichiro Toriwaki, “Virtual Pneumoperitoneum for Generating Virtual Laparoscopic Views Based on Volumetric Deformation”, MICCAI (Medical Image Computing and Computer-Assisted Intervention), 2004, P559-P567) may be applied to the gas injection simulation in which gas is injected through the anus.

In other words, the deformation processing unit 162 may perform the gas injection simulation based on the model of the non-gas injection state or the volume data, and generate the model of the virtual gas injection state or the volume data. The volume data obtained by capturing an image by the CT scanner 200 after the actual gas injection is performed or a model of the volume data may also be used. The gas injection simulation with changing gas injection amount may be performed based on the volume data obtained by capturing an image by the CT scanner 200 after actually gas is injected or the model based on the volume data. By the gas injection simulation, the operator can observe the state where gas is virtually injected by assuming that the subject PS is in a state where gas is injected without actually injecting gas into the subject PS. Of the gas injection states, a gas injection state estimated by the gas injection simulation may be referred to as a virtual gas injection state, and a state where gas is actually injected may be referred to as an actual gas injection state.

The gas injection simulation may be a large deformation simulation using the finite element method. In this case, the deformation processing unit 162 may segment the body surface containing the subcutaneous fat of the subject PS and an internal organ near the anus of the subject PS, via the region processing unit 161. The deformation processing unit 162 may model the body surface as a two-layer finite element of skin and body fat, and model the internal organ near the anus as a finite element, via the model setting unit 163. The deformation processing unit 162 may segment, for example, the rectum and bones in any manner, and add the segmented result to the model. A gas region may be provided between the body surface and the internal organ near the anus, and the gas injection region may be expanded (swollen) in response to the virtual gas injection. The gas injection simulation may not be performed.

The deformation processing unit 162 performs the registration processing based on the deformation of the model. The registration processing is processing to register the model of the subject in the virtual space with the position of the subject PS recognized by the surgical robot 300 in the actual space. In the registration processing, the coordinates of each point of the model of the subject generated in the preoperative simulation and the coordinates of each point of the subject PS in the actual space during surgery are matched. Accordingly, in the registration processing, the shape of the model of the subject and the shape of the subject PS are matched. Accordingly, the robotically-assisted surgical device 100 can match the position of the subject PS actually recognized by the surgical robot 300 with the position of the model of the subject PS, and can improve the accuracy of simulation and navigation using the model.

In the registration processing, each tissue included in the entire model of the subject may be registered with each tissue included in the entire subject PS. In the registration processing, the tissues included in a part of the model of the subject may be registered with the tissues included in a part of the subject PS. For example, the position of the intestinal wall in the model of the subject and the position of the intestinal wall in the subject PS in the actual space can be matched to be registered. When some of the tissues that the operator pays attention to can be registered, some other tissues may not have to be registered. The entire registration processing is performed in non-rigid registration. In the registration processing, instead of non-rigid registration, the deformation of the model (for example, a model of soft tissue) may be calculated independently and then rigid body registration may be performed. The non-rigid body registration may be registration according to the deformation of the model.

The deformation processing unit 162 may deform the model of the subject based on the contact position to the soft tissue detected by the contact sensor 60. The model of the subject may be deformed based on the contact position with the soft tissue and the reaction force from the soft tissue, which is detected by the contact sensor 60. Based on the actual endoscopic images, information on the deformation of soft tissues may be acquired by image analysis, and the model of the subject may be deformed based on the information on the deformation. The model of the subject which is a deformation target includes at least a model of the soft tissue which is a contact target. The timing for detecting the reaction force by the contact sensor 60 may be, for example, the timing while the contact sensor 60 is in contact with and presses the contact tissue and the contact position is changing or after the contact position is changed.

In the subject PS, the position of each tissue of the subject PS in the actual space can change depending on the body position change of the subject PS or surgery on the subject PS (for example, contact of the surgical instrument 30 with the tissue). In other words, the tissue of the subject PS can be deformed. The contact of the surgical instrument 30 with the tissue can include contact during organ movement and incision operations, for example. The deformation processing unit 162 deforms the model of the virtual space corresponding to such deformation of the tissue of the actual space.

The actual endoscopic image may also include soft tissues. One or a plurality of actual endoscopic images may be obtained. The deformation processing unit 162 may predict the image of the soft tissue at a predetermined section based on the model of the soft tissue. This predicted image is also referred to as a predicted image. The deformation processing unit 162 may analyze the actual endoscopic image and calculate the difference between the captured image of the soft tissue and the predicted image of the soft tissue. Then, the model of the soft tissue may be deformed based on this difference.

The tissue estimation unit 165 estimates the soft tissue (also referred to as contact target or contact tissue) with which the contact sensor 60 is in contact in the subject PS and the tissue (also referred to as back tissue) positioned behind this soft tissue. Soft tissue is, for example, the intestinal wall. The back tissue is, for example, soft tissue, hard tissue (for example, bone), or elastic tissue (for example, tendons, major arteries (for example, aorta, common iliac artery)). The tissue estimation unit 165 estimates the presence or absence of the back tissue, the type of back tissue, the position of the back tissue, and the like based on the contact position where the contact sensor 60 is in contact with the contact tissue or change in the contact position. A change in the contact position to the contact tissue can be described as deformation of the contact tissue. Deformation of tissues in the subject PS occurs, for example, when the body position is changed or when the surgical instrument 30 comes into contact with the tissue.

The tissue estimation unit 165 may also estimate the presence or absence of the back tissue, the type of back tissue, the position of the back tissue, and the like based on the contact position where the surgical instrument 30 is in contact with the contact tissue and the reaction force from the contact tissue. The tissue estimation unit 165 may also estimate the presence or absence of the back tissue, the type of back tissue, the position of the back tissue, and the like based on the contact position where the surgical instrument 30 is in contact with the contact tissue, the reaction force from the contact tissue, and the actual endoscopic image.

The image generation unit 166 generates various images. The image generation unit 166 generates a three-dimensional image or a two-dimensional image based on at least a part of the acquired volume data (for example, a region extracted in the volume data). The image generation unit 166 may generate a three-dimensional image or a two-dimensional image based on the volume data corresponding to the model or the like deformed by the deformation processing unit 162.

The display control unit 167 causes the display 130 to display various types of data, information, and images. The display control unit 167 displays an image (for example, a rendering image) generated by the image generation unit 166. The display control unit 167 may also adjust the brightness of the rendering image. The brightness adjustment may include, for example, adjustment of at least one of a window width (WW) and a window level (WL).

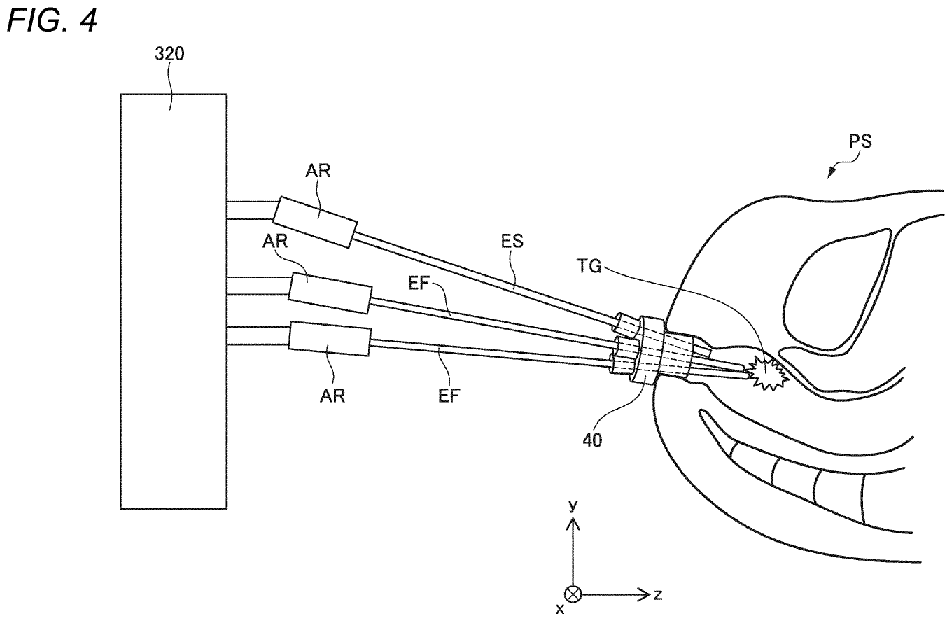

FIG. 4 is a view illustrating an example of a state of the inside of the platform 40, the surgical instrument 30, and the subject PS.

The end effector EF attached to the robot arm AR of the robot main body 320 is inserted into the subject PS through the platform 40. In FIG. 4, the platform 40 is installed on the anus, and reaches the target TG where the disease exists at a part of the rectum connected to the anus and the treatment is performed. The state near the target TG is imaged by the endoscope ES attached to the robot arm AR. Similar to the end effector EF, the endoscope ES is also inserted into the subject PS through the platform 40. The end effector EF for performing various treatments with respect to the target TO can be reflected on the actual endoscopic image.

The contact sensor 60 is attached to the distal end of the end effector EF. The contact sensor 60 comes into contact with the tissue (for example, target TG) in the subject PS and detects the contact position and reaction force. The robot main body 320 transmits the information on the contact position and reaction force detected by the contact sensor 60 to the robotically-assisted surgical device 100.

In FIG. 4, the x-direction, the y-direction, and the z-direction of the subject coordinate system (patient coordinate system) with respect to the subject PS are illustrated. The subject coordinate system is an orthogonal coordinate system. The x-direction may be along the left-right direction with respect to the subject PS. The y-direction may be the front-rear direction (thickness direction of the subject PS) with respect to the subject PS. The z-direction may be an up-down direction (the body axial direction of the subject PS) with respect to the subject PS. The x-direction, the y-direction, and the z-direction may be three directions defined by digital imaging and communications in medicine (DICOM).

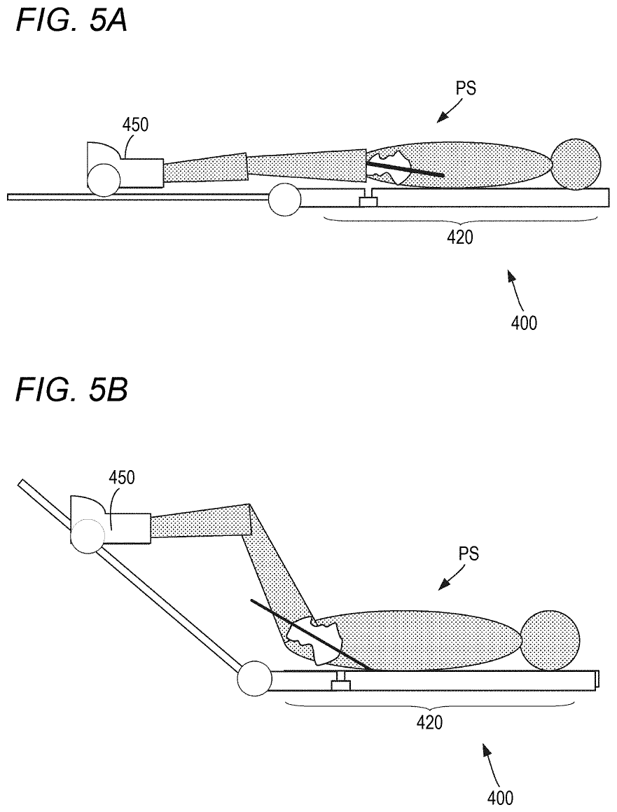

FIG. 5A is a view illustrating an example of a state of the pelvis 14 in a state where the body position of the subject PS is the lithotomy position and the leg part 31 is raised low. FIG. 5B is a view illustrating an example of a state of the pelvis 14 in a state where the body position of the subject PS is the lithotomy position and the leg part 31 is raised high.

The volume data is obtained, for example, by imaging the subject PS in a supine position using the CT scanner 200. The deformation processing unit 162 obtains information on the body position of the subject PS. Before the surgery on the subject PS, the deformation processing unit 162 may determine the body position (for example, lithotomy position) of the subject depending on the surgical procedure (for example, TAMIS) of the planned surgery on the subject PS. The deformation processing unit 162 may deform the model of the subject and perform the registration processing based on the volume data obtained in the supine position based on the information on the planned body position change (for example, change from supine position to lithotomy position).

The deformation processing unit 162 may also deform the model based on the measurement values of the various sensors included in the robotically-assisted surgical system 1 during surgery on the subject PS. For example, the deformation processing unit 162 may estimate the detailed body position (posture) of the subject PS based on the state of the pelvis 14 of the subject PS.

In FIGS. 5A and 5B, the subject PS is placed on a surgical bed 400. The surgical bed 400 has a table 420 on which the body part of the subject PS is placed, and a leg holding unit 450 that holds the leg part. The deformation processing unit 162 estimates the state of the pelvis 14 in the model of the subject according to, for example, the positional relationship between the table 420 and the leg holding unit 450, the model of the subject, and the kinematic model of the subject PS. The kinematic model has information, for example, on the length of the bones in the lower limb and the degrees of freedom of the joints in the lower limb of the subject PS. The positional relationship between the table 420 and the leg holding unit 450 may be calculated based on the detected values of various sensors included in the surgical bed 400. The various sensors may include sensors that detect the distance between the leg holding unit 450 and the table 420 and the angle of the leg holding unit 450 with respect to the table 420. The state of the pelvis 14 may include the position, orientation or movement of the pelvis 14 in the model of the subject. A position processing unit 164 may deform the model of the subject based on the state of the pelvis 14.

Next, an example of determining the target TG which is the surgery target and the tissue behind the target TG during surgery will be described.

During surgery, in a case where the target TG is an organ that is sequentially treated by the surgical instrument 30 before reaching the affected part, or the like, there are many cases where the target TG is positioned at the front and is captured by the endoscope ES. Therefore, the operator can observe the state of the target TG via the display 130 or the image display terminal 330. Meanwhile, there are many cases where tissues that exist behind the target TG are hidden behind the target TG and are difficult to confirm in the actual endoscopic images by the endoscope ES. In this case, the target TG is the contact tissue, and the tissue that exists behind the target TG is the back tissue.

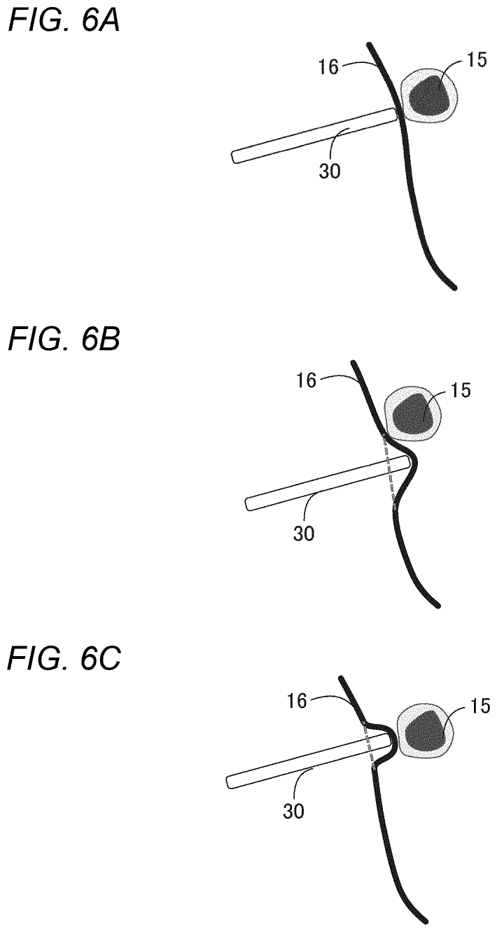

FIG. 6A is a schematic view illustrating an example in which there is a bone 15 behind an intestinal wall 16 with which the contact sensor 60 is in contact. In FIG. 6A, the intestinal wall 16 is an example of the contact tissue, and the bone is an example of the back tissue.

The tissue estimation unit 165 estimates that there is the bone 15 behind the intestinal wall 16 to be contacted as the target TG in a case where the change amount of the contact position where the surgical instrument 30 comes into contact with the intestinal wall 16 and moves is equal to a threshold value th1 (for example, matches the threshold value th1) and the reaction force from the intestinal wall 16 after the change in the contact position is equal to or greater than a threshold value th2. This estimation is based on the fact that the position of the bone 15 does not move even when the surgical instrument 30 comes into contact with the bone 15 through the intestinal wall 16. Accordingly, the tissue estimation unit 165 can estimate that there is the bone 15 in proximity to the intestinal wall 16, that is, the position of the back tissue. The tissue estimation unit 165 may estimate the back tissue only according to the change amount of the position of the contact tissue without considering the reaction force.

The threshold value th1 is the length corresponding to the thickness of the contact tissue (here, the intestinal wall 16). Information on the thickness of the contact tissue may be obtained, for example, from the thickness of the model (for example, an intestinal wall model) of the contact tissue in the model and set to the threshold value th1. The threshold value th2 is a threshold value for detecting hard tissue such as the bone 15. For example, the reaction force from the bone 15 through the intestinal wall 16 may be measured in advance, and the reaction force may be set to the threshold value th2. The setting of the threshold values th1 and th2 may be performed by the tissue estimation unit 165.

FIG. 6B is a schematic view illustrating an example in which there is no bone 15 behind the intestinal wall 16 with which the contact sensor 60 is in contact. In FIG. 6B, the intestinal wall 16 is an example of the contact tissue, and the bone 15 is an example of the back tissue.

The tissue estimation unit 165 estimates that there is no bone 15 behind the intestinal wall 16 to be contacted as the target TG in a case where the change amount of the contact position where the surgical instrument 30 comes into contact with the intestinal wall 16 and moves is greater than the threshold value th1 and the reaction force from the intestinal wall 16 after the change in the contact position is less than the threshold value th2. This estimation is based on the fact that the intestinal wall 16 moves largely without the tissue for stopping the movement of the surgical instrument 30 behind the intestinal wall 16. The tissue estimation unit 165 may estimate the back tissue only according to the change amount of the position of the contact tissue without considering the reaction force.

FIG. 6C is a schematic view illustrating an example in which there is the bone 15 a little apart behind the intestinal wall 16 with which the contact sensor 60 is in contact. In FIG. 6C, the intestinal wall 16 is an example of the contact tissue, and the bone 15 is an example of the back tissue.

The tissue estimation unit 165 estimates that there is the bone 15 apart from the intestinal wall 16 behind the intestinal wall 16 contacted as the target TG in a case where the change amount of the contact position where the surgical instrument 30 comes into contact with the intestinal wall 16 and moves is greater than the threshold value th1 and the reaction force from the intestinal wall 16 after the change in the contact position is equal to or greater than the threshold value th2. This estimation is based on the fact that the intestinal wall 16 is movable to a certain extent, but does not move once the intestinal wall 16 reaches the bone 15. The tissue estimation unit 165 may estimate that the difference between the change amount of contact position and the thickness (corresponding to the threshold value th1) of the intestinal wall 16 is the distance between the intestinal wall 16 and the bone 15. The tissue estimation unit 165 may estimate the back tissue only according to the change amount of the position of the contact tissue without considering the reaction force. In this manner, the tissue estimation unit 165 can estimate the position of the back tissue.

The tissue estimation unit 165 may estimate that there is the bone 15 apart from the intestinal wall 16 behind the intestinal wall 16 contacted as the target TG in a case where the change amount of the contact position where the surgical instrument 30 comes into contact with the intestinal wall 16 and moves is the length corresponding to the sum of the thickness of the intestinal wall 16 and the distance between the intestinal wall 16 and the bone 15. The information on the distance between the intestinal wall 16 and the bone 15 may be acquired from the distance between the intestinal wall model and the bone model in the model.

In a case of FIGS. 6A, 6B, and 6C, since the intestinal wall 16 is actually deformed by the contact of the surgical instrument 30, the deformation processing unit 162 deforms at least the intestinal wall model in the model according to the change amount of the contact position. The robotically-assisted surgical device 100 can recognize that there is the bone 15 which is behind the intestinal wall 16 and cannot be seen.

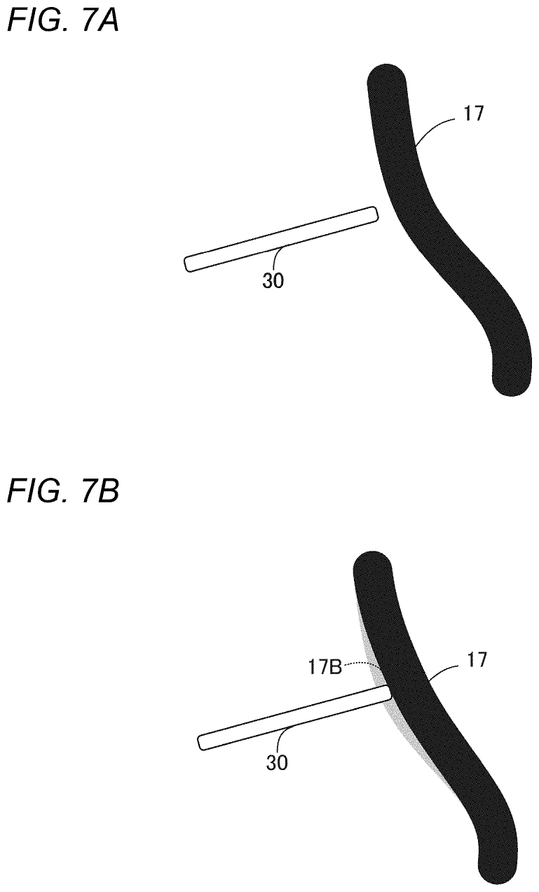

FIG. 7A is a schematic view illustrating an example of a state before the contact sensor 60 comes into contact with a tendon 17 with which the contact sensor 60 is in contact. FIG. 7B is a schematic view illustrating an example of a state where the contact sensor 60 is in contact with the tendon 17 with which the contact sensor 60 is in contact. In FIGS. 7A and 7B, the tendon 17 is an example of a contact tissue.

The tissue estimation unit 165 estimates that the contact sensor 60 is in contact with an elastic tissue (here, tendon 17) in a case where the change amount of the contact position where the surgical instrument 30 comes into contact with the tendon 17 and moves is equal to or greater than a threshold value th11 and the reaction force from the intestinal wall 16 after the change in the contact position is equal to or greater than a threshold value th12. This estimation is based on the fact that the tendon 17 has elasticity and the tendon 17 moves largely without the tissue for stopping the movement of the surgical instrument 30 behind the tendon 17.

The threshold value th11 is a length longer than the thickness of the contact tissue (here, tendon 17), taking into account elasticity. Therefore, the threshold value th11 is a value greater than the threshold value th1 and it is assumed that the contact tissue is somewhat elongated. The threshold value th12 is a threshold value for detecting tissues that are softer and more elastic than hard tissues. Therefore, the threshold value th12 is a value less than the threshold value th2. The setting of the threshold values th11 and th12 may be performed by the tissue estimation unit 165.

The tissue estimation unit 165 also acquires the actual endoscopic image captured by the endoscope ES. The tissue estimation unit 165 may perform image analysis on the actual endoscopic image to determine the type of tissue (for example, the bone 15, the intestinal wall 16, and the tendon 17) with which the contact sensor 60 is in contact. Here, the tissue estimation unit 165 determines that the contact sensor 60 is in contact with the tendon 17.

In FIG. 7B, since the tendon 17 is actually deformed by the contact of the surgical instrument 30, the deformation processing unit 162 deforms at least the model of the tendon in the model according to the change amount of the contact position. In a case where the surgical instrument 30 presses the tendon 17 in this manner, the tendon 17 deforms over a wide range (the deformation amount is large) because the tendon 17 is an elastic tissue. In this case, since the tendon 17 deforms the surgical instrument 30 in the pressing direction, there is not much change on the actual endoscopic image, and thus, it is difficult to use the image for registration. In contrast, the robotically-assisted surgical device 100 can perform registration processing based at least on changes in the contact position. The reference character “17B” in FIG. 7B indicates the tendon before deformation.

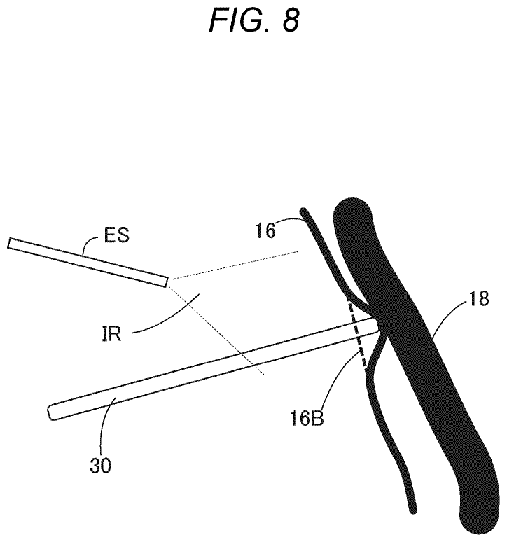

FIG. 8 is a schematic view illustrating an example in which there is a major artery 18 behind the intestinal wall 16 with which the contact sensor 60 is in contact. In FIG. 8, the intestinal wall 16 is an example of the contact tissue, and the major artery 18 is an example of the back tissue. The major artery 18 is, for example, the aorta and common iliac artery.

The tissue estimation unit 165 estimates that there is an elastic tissue, such as the major artery 18, behind the intestinal wall 16 to be contacted as the target TG in a case where the change amount of the contact position where the surgical instrument 30 comes into contact with the intestinal wall 16 and moves is equal to or greater than the threshold value th1 and the reaction force from the intestinal wall 16 after the change in the contact position is equal to or greater than the threshold value th12. This estimation is based on the fact that the major artery 18 does not move much and is easily subjected to the reaction force from the major artery 18 through the intestinal wall 16 when the surgical instrument 30 comes into contact with the major artery 18 through the intestinal wall 16.

The tissue estimation unit 165 may acquire the actual endoscopic image. The tissue estimation unit 165 may perform image analysis on the actual endoscopic image to determine the type of tissue (for example, the bone 15, the intestinal wall 16, and the tendon 17) with which the contact sensor 60 is in contact. Here, the tissue estimation unit 165 determines that the contact sensor 60 is in contact with the intestinal wall 16.

In a case of FIG. 8, since the intestinal wall 16 is actually deformed by the contact of the surgical instrument 30, the deformation processing unit 162 deforms at least the intestinal wall model in the model according to the change amount of the contact position. When the intestinal wall 16 is pressed in a case where there is the major artery 18 behind the contact tissue, the major artery 18 and the intestinal wall 16 deform together. After the change in contact position, there are cases where the reaction force from the major artery 18 as the back tissue obtained through the intestinal wall 16 may increase, the advancing direction of the surgical instrument 30 in the actual endoscopic image is shifted from the direction of actually being pressed, or the surgical instrument 30 bends. Based on these changes in the reaction force or the image, the tissue estimation unit 165 can recognize that the back tissue is an elastic tissue. The reference character “18B” in FIG. 8 indicates the intestinal wall before deformation, and the reference character “IR” indicates the imaging range (visual field) by the endoscope ES.

In a case where the back tissue is an elastic tissue, the tissue estimation unit 165 can recognize that a dangerous part that requires attention during surgery, such as the major artery 18, is hidden behind the soft tissue which is the contact tissue. The dangerous part hidden behind is not drawn in the actual endoscopic image. In this case, the display control unit 167 may display warning information indicating that there is a dangerous part, on the display 130 or the image display terminal 330. Accordingly, the operator and those involved in the surgery other than the operator can be informed of the presence of the dangerous part. The display of the dangerous part is one example of the presentation of the dangerous part, and the warning information indicating that there is the dangerous part may be presented by other presentation methods (for example, voice output, vibration). The information on which tissue is the dangerous part may be held in the memory 150.

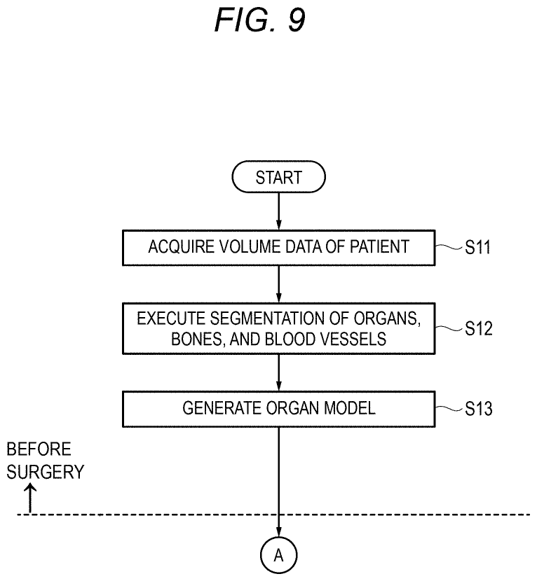

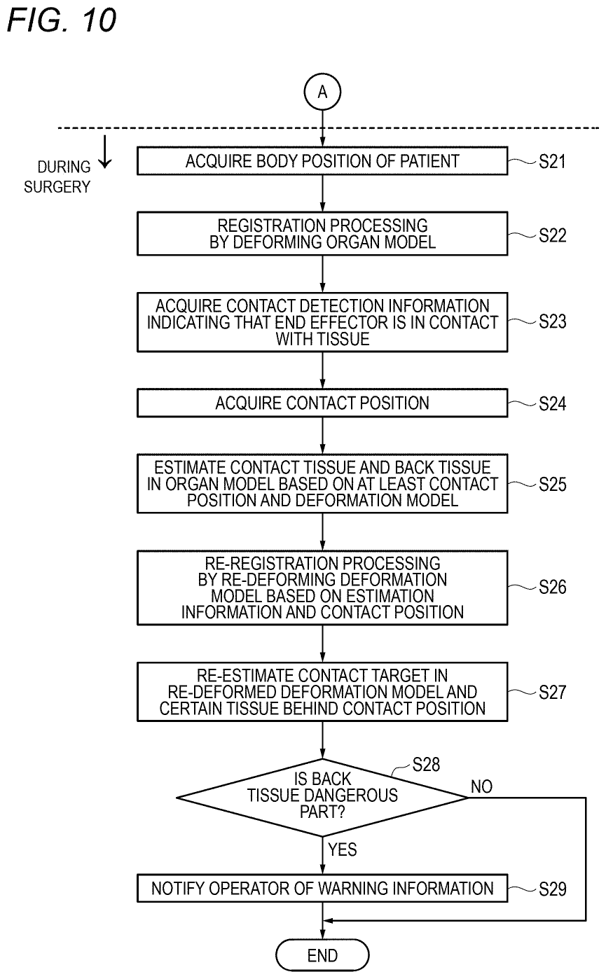

FIGS. 9 and 10 are flowcharts illustrating an operation example of the robotically-assisted surgical device 100. S11 to S13 in FIG. 9 are executed, for example, before surgery, and S21 to S29 in FIG. 10 are executed, for example, during surgery. Each processing here is executed by each part of the processing unit 160. Here, the use of an organ model of the rectum is described, but other models may be used.

First, before surgery, the volume data of the subject PS (for example, a patient) is acquired (S11). Segmentation to extract regions of organs, bones, and blood vessels is executed (S12). The organ model of the rectum is generated based on the volume data (S13).

When the robotic surgery is started, the surgical robot 300 and the surgical bed 400 on which the subject PS is placed are arranged at a predetermined position. During surgery, the surgical instrument 30 is inserted into the subject PS via the platform 40 installed on the anus.

Then, the body position (detailed body position) of the subject PS is acquired (S21). For example, the body position of the subject PS may be determined by being designated by the operator via the UI 120. The body position of the subject PS may be determined according to the form of the deformable surgical bed 400. The body position of the subject PS may also be determined according to the surgical procedure.

Based on the acquired body position of the subject PS, the organ model is deformed and registered (S22). The change in the body position of the subject PS may be acquired, and the organ model may be deformed and registered based on the change in the body position. By the deformation of the organ model, the registration is performed by matching the position of the organ model of the rectum in the virtual space and the position of the rectum in the actual space recognized by the surgical robot 300.

The operator operates the surgical instrument 30 via the surgical robot 300, inserts the surgical instrument 30 (for example, the end effector EF and the endoscope ES) into the subject PS, and performs various treatments. At this time, the contact sensor 60 detects that the end effector EF is in contact with the contact tissue. The tissue estimation unit 165 acquires the contact detection information indicating that the end effector EF is in contact with the contact tissue, from the surgical robot 300 (S23). The contact detection information may include information on the contact position where the end effector EF is in contact with the contact tissue. The contact detection information may include information on the reaction force received from the contact tissue.

The tissue estimation unit 165 acquires the contact position and reaction force information included in the contact detection information from the surgical robot 300 (S24). The actual endoscopic image may be acquired from the surgical robot 300. Based on at least the contact position or the change amount in the contact position and the organ model, the contact tissue in the organ model and the back tissue behind the contact tissue are estimated (S25). In this case, the contact tissue and the type of back tissue (for example, rectum, bone, blood vessel, tendon) are estimated. In this case, the contact tissue and the back tissue in the organ model may be estimated based on the contact position or the change amount of the contact position, the reaction force received from the contact tissue, and the organ model. The contact tissue and the back tissue in the organ model may be estimated based on the contact position or the change amount of the contact position, the reaction force received from the contact tissue, the actual endoscopic image, and the organ model.

In a case where the difference between the position of the organ model of the rectum and the position of the rectum in the actual space is small, the contact tissue and the back tissue in the organ model are the same as the contact tissue and the back tissue of the rectum in the actual space. Meanwhile, in a case where the difference between the position of the organ model of the rectum and the position of the rectum in the actual space is large, the contact tissue and the back tissue in the organ model are different from the contact tissue and the back tissue of the rectum in the actual space.

Based on the estimated contact tissue and back tissue (that is, estimated information) and the contact position, the registration processing is performed by re-deforming the organ model (S26). In this case, the registration processing may be performed by extracting the estimated regions of the contact tissue and the back tissue from the organ model, and by deforming the extracted regions. In this manner, after the registration processing is performed corresponding tissue deformation based on the body position of the subject PS, the registration processing may be performed corresponding to tissue movement or deformation caused by contact with some tissue in the subject PS. The registration processing based on contact with the tissue of the subject PS may be performed without the registration processing based on the body position of the subject PS.

The contact tissue and the back tissue in the re-deformed organ model is re-estimated (S27). In this case, the contact tissue and the type of back tissue (for example, rectum, bone, blood vessel, tendon) are re-estimated. The information used for re-estimation may be the same as the information used for estimation in S25. As the organ model is re-deformed in S26, the position of each point in the organ model is changed. Meanwhile, the contact position detected by the contact sensor 60 does not change. Therefore, the result of the re-estimation of the contact tissue and the back tissue can be different from that of the estimation in S25.

It is determined whether or not the re-estimated back tissue is a dangerous part (S28). The information on the dangerous part is held in the memory 150 and may be referred to as appropriate. For example, the dangerous part is a major artery (for example, the aorta).

In a case where the re-estimated back tissue is a dangerous part, the warning information indicating that the back tissue is a dangerous part is displayed (S29). in a case where it is determined whether or not the contact tissue is a dangerous part, and the contact tissue is a dangerous part, the warning information indicating that the contact tissue is a dangerous part is displayed.

The processing of S21 to S29 may be repeated during surgery. At least a part of the processing of S11 to S13 may be repeated by imaging the patient with a cone beam CT or the like during surgery.

In this manner, the robotically-assisted surgical device 100 takes the contact of the surgical instrument 30 with the soft tissue as an opportunity to perform the registration processing based on the contact position and the deformation of the soft tissue. Accordingly, the robotically-assisted surgical device 100 can perform the registration processing by bringing the surgical instruments 30 into contact with the soft tissues such as the intestinal wall, even when the surgical instrument 30 cannot directly come into contact with hard tissues such as bones that will serve as a reference for registration. Therefore, the position of the subject PS in the actual space recognized by the surgical robot 300 and the position of the model of the subject in the virtual space are matched, and thus, the robotically-assisted surgical device 100 can improve the accuracy of simulation and navigation using the model. The robotically-assisted surgical device 100 can also determine the type of the back tissue behind the contact tissue, and thus, the following events can be suppressed even when the back tissue is not reflected in the actual endoscopic image by the endoscope ES. As a specific example, as the surgical instrument 30 continues to press the intestinal wall at the front, the surgical instrument 30 reaches the bone at the back through the intestinal wall, and is sandwiched between the surgical instrument 30 and the bone, and it is possible to suppress penetration of the surgical instrument 30 through the intestinal wall. Accordingly, the robotically-assisted surgical device 100 contributes to safety in robotic surgery.

As a comparative example, it is assumed that the contact position of the hard tissue is detected instead of the contact position of the soft tissue, and the registration processing is based on this contact position. The hard tissue does not deform even when the contact sensor 60 is in contact therewith, for example, the bones are fixed in orthopedic surgery, and thus, it is not assumed that the hard tissue does not move after the registration processing. In contrast, the robotically-assisted surgical device 100 can be moved, rotated, or deformed many times during surgery because the surgical instrument 30 is in contact with soft tissues. Even in this case, the robotically-assisted surgical device 100 can register the subject PS and the model by deforming the model in accordance with each deformation of the tissue in the subject PS. In the field of orthopedic surgery, which deals with hard tissues, a high accuracy is required as the registration accuracy, but in fields other than orthopedic surgery, which deals with soft tissues, the registration accuracy may be somewhat lower, for example, may be within a range of an error of 3 mm or less. In the embodiment, the target of various treatments in surgery may be the soft tissue which is the contact target, and the back tissue such as bones or major blood vessels may not have to be the surgery target.

The robotically-assisted surgical device 100 can perform the registration processing when the surgical instrument 30 is in contact with the soft tissue, taking into account the deformation of the soft tissue and the hard back tissue. The registration processing is executed at least in the depth direction in a case where the endoscope ES is the viewpoint. In the direction perpendicular to the depth direction (that is, the direction along the image surface of the actual endoscopic image), the registration processing may not have to be performed. This is because it is possible to confirm the up-down and left-right direction in the image by observing the actual endoscopic image captured by the endoscope ES. Accordingly, the robotically-assisted surgical device 100 can assist the implementation of each surgical treatment with full consideration of the depth direction. Even in a case where the display of the endoscope ES is not a 3D display with sense of depth, the information in the depth direction increases, and accordingly, the safety of the operation can be improved.

Although various embodiments have been described above with reference to the drawings, it is needless to say that the present disclosure is not limited to such examples. It is clear that a person skilled in the art can come up with various changes or modifications within the scope of the claims, and it is understood that these changes or modifications naturally belong to the technical scope of the present disclosure.

For example, the contact sensor 60 is illustrated as a contact detection unit that detects contact of the surgical instrument 30 with soft tissues, but this is not limited thereto. For example, known contact detection techniques related to the haptic feedback, such as those illustrated in Reference Non-Patent Literature 2 (Allison M. Okamura, “Haptic Feedback in Robot-Assisted Minimally Invasive Surgery”, searched on Mar. 3, 2020, Internet <URL: https://www.ncbi.nlm.nih.gov/pmc/articles/PMC2701448/>), may be used.

For example, an ultrasound probe may be used to detect contact with a soft tissue. The deformation processing unit 162 may recognize the bending of the surgical instrument 30 and the deformation of the contact tissue based on the image analysis on the actual endoscopic image captured by the endoscope ES. Then, based on the bending of the surgical instrument 30 and the deformation of the tissue, the contact of the surgical instrument 30 with the tissue may be detected.

As an example of detecting the distal end position of the surgical instrument 30, an example is illustrated in which the contact position is detected by the contact sensor 60 installed at the distal end of the surgical instrument 30, but the disclosure is not limited thereto. For example, the deformation processing unit 162 may acquire the angle information detected by the angle detector installed in the robot main body 320 and the information on the kinematics of the robot main body 320. The deformation processing unit 162 may detect the distal end position of the surgical instrument 30 based on this angular information and the information on the kinematics of the robot main body 320. The deformation processing unit 162 may also detect the distal end position of the surgical instrument 30 based on the insertion distance information indicating the insertion distance of the surgical instrument 30 into the subject PS described above. The deformation processing unit 162 may also detect the distal end position of the surgical instrument 30 and the deformation of the neighboring tissue in the vicinity of the surgical instrument 30 based on image analysis with respect to the actual endoscopic image. This position may be the distal end position of the surgical instrument 30 with respect to the position of the endoscope ES. In a case where the distal end of the surgical instrument 30 is in contact with the soft tissue, the distal end of the surgical instrument 30 corresponds to the contact position. Accordingly, the distal end position of the surgical instrument 30 when being in contact with the soft tissue may be used to deform the model.

Although an example is illustrated in which the reaction force is detected by the contact sensor 60, but the disclosure is not limited thereto. For example, the deformation processing unit 162 may recognize soft tissue distortions based on image analysis with respect to the actual endoscopic image and estimate the reaction force received from the soft tissue based on the state of the distortion (for example, shape, size).

An example is illustrated in which the contact sensor 60 is installed in at least one of the plurality of surgical instruments 30, but the disclosure is not limited thereto. For example, a simple rod may be attached to the robot arm AR, and the contact sensor 60 may be attached to the distal end of this rod. This rod extends the robot arm AR and may be attached instead of the surgical instrument 30.

An example is illustrated in which the contact sensor 60 comes into contact with the soft tissue in the subject PS via the platform 40, but the disclosure is not limited thereto. For example, the contact sensor 60 may be in direct contact with the body surface of the subject PS.

The endoscope ES is not limited to hard part endoscopes, but can also be a soft part endoscope.

Although the above-described embodiments can be applied to TAMIS, the embodiment may be applied to other surgical procedures, for example, to transanal total mesenteric excision (TaTME). The embodiments may also be applied to single-hole laparoscopic surgery.

The embodiments can be used not only for the robotic surgery based on the operation of the operator, but also for autonomous robotic surgery (ARS) or semi-ARS. ARS is a fully automatic robotic surgery performed by an AI-equipped surgical robot. Semi-ARS basically automatically performs the robotic surgery by an AI-equipped surgical robot, and partially performs the robotic surgery by the operator.

Although the endoscopic surgery by the robotic surgery is exemplified, the surgery may be performed by the operator directly operating the surgical instrument 30. In this case, the robot main body 320 may be the operator, the robot arm AR may be the arm of the operator, and the surgical instrument 30 may be forceps and an endoscope that the operator grasps and uses for treatment.

Although an example is illustrated in which the endoscopic surgery performs robotic surgery, but the endoscopic surgery may be robotic surgery performed by direct visual inspection by the operator. The endoscopic surgery may be also be robotic surgery using a camera that is not inserted into the patient. In this case, the robot can be operated by the operator or by an assistant.

The preoperative simulation and the intraoperative navigation may be configured by a separate robotically-assisted surgical device. For example, the preoperative simulation may be performed by a simulator, and the intraoperative navigation may be performed by a navigator.

The robotically-assisted surgical device 100 may include at least the processor 140 and the memory 150. The transmission/reception unit 110, the UI 120, and the display 130 may be externally attached to the robotically-assisted surgical device 100.

It is exemplified that the volume data as the captured CT image is transmitted from the CT scanner 200 to the robotically-assisted surgical device 100. Instead of this, the volume data may be transmitted to and stored in a server (for example, an image data server (PACS) (not illustrated)) or the like on the network such that the volume data is temporarily stored. In this case, the transmission/reception unit 110 of the robotically-assisted surgical device 100 may acquire the volume data from a server or the like via a wired circuit or a wireless circuit when necessary, or may acquire the volume data via any storage medium (not illustrated).

It is exemplified that the volume data as the captured CT image is transmitted from the CT scanner 200 to the robotically-assisted surgical device 100 via the transmission/reception unit 110. This also includes a case where the CT scanner 200 and the robotically-assisted surgical device 100 are established by being substantially combined into one product. This also includes a case where the robotically-assisted surgical device 100 is handled as the console of the CT scanner 200. The robotically-assisted surgical device 100 may be provided in the surgical robot 300.

Although it is exemplified that the CT scanner 200 is used to capture an image and the volume data including information on the inside of the subject is generated, the image may be captured by another device to generate the volume data. Other devices include a magnetic resonance imaging (MRI) device, a positron emission tomography (PET) device, a blood vessel imaging device (angiography device), or other modality devices. The PET device may be used in combination with other modality devices.

A robotically-assisted surgical method in which the operation in the robotically-assisted surgical device 100 is defined can be visualized. A program for causing a computer to execute each step of the robotically-assisted surgical method can be visualized.

### Overview of Above-Described Embodiment

According to one aspect of the above-described embodiment, the robotically-assisted surgical device 100 that assists the robotic surgery by the surgical robot 300 includes the processing unit 160. The processing unit 160 has a function of acquiring 3D (for example, model, volume data) data of the subject PS, acquiring a contact position where the surgical instrument 30 provided in the surgical robot 300 is in contact with a soft tissue of the subject PS, acquiring firmness (for example, reaction force) of the contact position of the soft tissue of the subject PS, and performing registration of a position of the 3D data with a position of the subject PS recognized by the surgical robot 300 according to deformation of the soft tissue in the 3D data, based on the contact position and the firmness.

Accordingly, the robotically-assisted surgical device 100 can register the subject PS in the actual space with the 3D data corresponding to the subject PS in the virtual space based on the results of contact with the soft tissue which is the contact tissue, even in a case where the hard tissue, which is easily used as a reference for registration, is the back tissue, the hard tissue cannot be confirmed by the actual endoscopic image, and it is not possible to directly come into contact with the hard tissue. Accordingly, the robotically-assisted surgical device 100 can easily perform registration by reflecting the deformation of the tissues in the 3D data even in a case of tissues that are easily moved in the subject PS, such as soft tissues. In this manner, the actual position of the subject PS and the position of the 3D data of the subject can be easily registered, taking into account the soft tissues that are easily deformed. Accordingly, even in a case of the robotically-assisted surgical device 100 with poor sense of touch, the operator can grasp a certain tissue behind the soft tissue.

The processing unit 160 acquires at least one actual endoscopic image (one example of the captured image) which is captured by an endoscope that images the inside of the subject PS and includes the soft tissue, analyzes the actual endoscopic image and calculates a difference between a predicted image of the soft tissue, which is predicted based on the soft tissue in the acquired 3D data, and the captured image of the soft tissue, deforms the soft tissue in the 3D data based on the difference, and performs the registration based on the deformation of the soft tissue in the 3D data. Accordingly, the robotically-assisted surgical device 100 can perform registration taking into account events (for example, the advancing direction or bending of the surgical instrument 30) that can be grasped from the actual endoscopic image through image analysis or the like.

Based on the contact position and firmness, the processing unit 160 may estimate whether or not there is a bone behind the soft tissue with which the surgical instrument 30 is in contact from the perspective of the surgical instrument 30. Accordingly, the robotically-assisted surgical device 100 can recognize the presence or absence of the bone as the back tissue based on the results of contact with the soft tissue. Therefore, for example, the robotically-assisted surgical device 100 can instruct the surgical robot 300 to reduce the force that comes into contact with the soft tissue, or can instruct the upper limit value of the force that comes into contact with the soft tissue based on the presence of the bone, and it is possible to improve the safety of robotic surgery.

The processing unit 160 may estimate whether or not surgical instrument 30 is in contact with the elastic tissue based on the contact position and firmness. Accordingly, the robotically-assisted surgical device 100 can recognize the presence of the elastic tissue as the contact tissue based on the results of contact with the contact tissue. Therefore, for example, the robotically-assisted surgical device 100 can instruct the surgical robot 300 to reduce the force that comes into contact with the elastic tissue, or can instruct the upper limit value of the force that comes into contact with the elastic tissue, and it is possible to improve the safety of robotic surgery.

Based on the contact position and firmness, the processing unit 160 may estimate whether or not there is the elastic tissue behind the soft tissue with which the surgical instrument 30 is in contact from the perspective of the surgical instrument 30. Accordingly, the robotically-assisted surgical device 100 can recognize the presence or absence of the elastic tissue as the back tissue based on the results of contact with the soft tissue. Therefore, for example, the robotically-assisted surgical device 100 can instruct the surgical robot 300 to reduce the force that comes into contact with the soft tissue, or can instruct the upper limit value of the force that comes into contact with the soft tissue based on the presence of the elastic tissue, and it is possible to improve the safety of robotic surgery.

Based on the contact position and firmness, the processing unit 160 may determine whether or not there is a dangerous part behind the soft tissue with which the surgical instrument 30 is in contact from the perspective of the surgical instrument 30. In a case where the processing unit 160 determines that there is a dangerous part, the warning information indicating that there is the dangerous part may be presented. Accordingly, the operator can confirm the presence of a dangerous part as a back tissue by confirming the presentation of the warning information. Therefore, for example, when operating the robot operation terminal 310, the operator can, for example, pay close attention when the surgical instrument 30 approaches the neighborhood of the contact tissue.

According to one aspect of the above-described embodiment, the surgical robot 300 includes the robot arm AR, the surgical instrument 30 attached to the robot arm AR, and the processing unit 35. The processing unit 35 acquires the contact position where the surgical instrument 30 is in contact with the soft tissue of the subject PS, acquires the firmness of the contact position of the soft tissue of the subject PS, and transmit the contact position and firmness information to the robotically-assisted surgical device 100 for assisting robotic surgery by the surgical robot 300.

Accordingly, the surgical robot 300 can acquire information on the contact position in soft tissue and the firmness received by the soft tissue, and thus, it is possible to register the subject PS in the actual space with the 3D data corresponding to the subject PS in the virtual space by the robotically-assisted surgical device 100.

According to another aspect of the above-described embodiment, there is provided a robotically-assisted surgical method that assists endoscopic surgery by the surgical robot 300, including: acquiring 3D data of the subject PS; acquiring a contact position where the surgical instrument 30 provided in the surgical robot 300 is in contact with a soft tissue of the subject PS; acquiring firmness of the contact position of the soft tissue of the subject PS; and performing registration of a position of the 3D data with a position of the subject PS recognized by the surgical robot 300 according to deformation of the soft tissue in the 3D data, based on the contact position and the firmness.

According to still another aspect of the embodiment, there is provided a program for causing a computer to execute the above-described robotically-assisted surgical method.

In view of the above-described circumstances, the present disclosure provides a robotically-assisted surgical device, a surgical robot, a robotically-assisted surgical method, and a program that can easily register the actual position of the subject with the position of the model of the subject, taking into account soft tissues that are easily deformed.

1. A robotically-assisted surgical device that assists robotic surgery by a surgical robot, the robotically-assisted surgical device comprising:
a processor, wherein
the processor is configured to:
acquire 3D data of a subject;
acquire a contact position where a surgical instrument provided in the surgical robot is in contact with a soft tissue of the subject;
acquire firmness of the contact position of the soft tissue of the subject; and
perform registration of a position of the 3D data with a position of the subject recognized by the surgical robot according to deformation of the soft tissue in the 3D data, based on the contact position and the firmness.

2. The robotically-assisted surgical device according to claim 1, wherein
the processor is configured to:
acquire at least one captured image which includes the soft tissue and is captured by an endoscope configured to image an inside of the subject;
analyze the captured image and calculate a difference between a predicted image of the soft tissue, which is predicted based on the soft tissue in the acquired 3D data, and the captured image of the soft tissue;
deform the soft tissue in the 3D data based on the difference; and
perform the registration based on the deformation of the soft tissue in the 3D data.

3. The robotically-assisted surgical device according to claim 1, wherein
the processor is configured to estimate whether or not there is a bone behind the soft tissue with which the surgical instrument is in contact, based on the contact position and firmness.

4. The robotically-assisted surgical device according to claim 1, wherein
the processor is configured to estimate whether or not the surgical instrument is in contact with an elastic tissue based on the contact position and the firmness.

5. The robotically-assisted surgical device according to claim 1, wherein
the processor is configured to determine whether or not there is an elastic tissue behind the soft tissue with which the surgical instrument is in contact from a viewpoint of the surgical instrument, based on the contact position and the firmness.

6. The robotically-assisted surgical device according to claim 4, wherein
the elastic tissue is a blood vessel.

7. The robotically-assisted surgical device according to claim 1, wherein
the processor is configured to:
determine whether or not there is a dangerous part behind the soft tissue with which the surgical instrument is in contact from the viewpoint of the surgical instrument, based on the contact position and the firmness; and
show warning information indicating that there is the dangerous part, in a case where it is determined that there is the dangerous part.

8. The robotically-assisted surgical device according to claim 1, wherein
the robotic surgery is endoscopic surgery.

9. A surgical robot that assists surgery, comprising:
a robot arm;
a surgical instrument attached to the robot arm; and
a processor, wherein
the processor is configured to:
acquire a contact position where the surgical instrument is in contact with a soft tissue of a subject;
acquire firmness of the contact position of the soft tissue of the subject; and
transmit information on the contact position and the firmness to a robotically-assisted surgical device that assists robotic surgery by the surgical robot.

10. A robotically-assisted surgical method that assists endoscopic surgery by a surgical robot, the robotically-assisted surgical method comprising:
acquiring 3D data of a subject;
acquiring a contact position where a surgical instrument provided in the surgical robot is in contact with a soft tissue of the subject;
acquiring firmness of the contact position of the soft tissue of the subject; and
performing registration of a position of the 3D data with a position of the subject recognized by the surgical robot according to deformation of the soft tissue in the 3D data, based on the contact position and the firmness.

11. The robotically-assisted surgical method according to claim 10, comprising:
acquiring at least one captured image which includes the soft tissue and is captured by an endoscope configured to image an inside of the subject;
analyzing the captured image and calculate a difference between a predicted image of the soft tissue, which is predicted based on the soft tissue in the acquired 3D data, and the captured image of the soft tissue; and
deforming the soft tissue in the 3D data based on the difference; wherein
the performing the registration is performed based on the deformation of the soft tissue in the 3D data.

12. The robotically-assisted surgical method according to claim 1, comprising
estimating whether or not there is a bone behind the soft tissue with which the surgical instrument is in contact, based on the contact position and firmness.

13. A system comprising:
a surgical robot; and
the robotically-assisted surgical device according to claim 1.

14. The system according to claim 13, wherein
the processor is configured to:
acquire at least one captured image which includes the soft tissue and is captured by an endoscope configured to image an inside of the subject;
analyze the captured image and calculate a difference between a predicted image of the soft tissue, which is predicted based on the soft tissue in the acquired 3D data, and the captured image of the soft tissue;
deform the soft tissue in the 3D data based on the difference; and
perform the registration based on the deformation of the soft tissue in the 3D data.

15. The system according to claim 13, wherein
the processor is configured to estimate whether or not there is a bone behind the soft tissue with which the surgical instrument is in contact, based on the contact position and firmness.

16. The system according to claim 13, wherein
the processor is configured to estimate whether or not the surgical instrument is in contact with an elastic tissue based on the contact position and the firmness.

17. The system according to claim 13, wherein
the processor is configured to determine whether or not there is an elastic tissue behind the soft tissue with which the surgical instrument is in contact from a viewpoint of the surgical instrument, based on the contact position and the firmness.

18. The system according to claim 16, wherein
the elastic tissue is a blood vessel.

19. The system according to claim 13, wherein
the processor is configured to:
determine whether or not there is a dangerous part behind the soft tissue with which the surgical instrument is in contact from the viewpoint of the surgical instrument, based on the contact position and the firmness; and
show warning information indicating that there is the dangerous part, in a case where it is determined that there is the dangerous part.

20. The system according to claim 13, wherein
the robotic surgery is endoscopic surgery.
